(19) United States
(12) Patent Application Publication
(10) Pub. No.: US 2021/0186937 A1
(43) Pub. Date: Jun. 24, 2021

(54) COMPOSITIONS AND METHODS FOR TREATING NEURODEGENERATIVE DISORDERS WITH RIFAXIMIN

(71) Applicant: Duke University, Durham, NC (US)

(72) Inventors: Paul SUHOCKI, Durham, NC (US); Pudugramam DORAISWAMY, Durham, NC (US)

(21) Appl. No.: 17/057,541

(22) PCT Filed: May 22, 2019
(86) PCT No.: PCT/US2019/033552

§ 371 Date: Nov. 20, 2020

Related U.S. Application Data
(60) Provisional application No. 62/674,783, filed on May 22, 2018.

(51) Int. Cl. A61K 31/437; A61P 25/28

(52) U.S. Cl. CPC A61K 31/437; A61P 25/28

(57) ABSTRACT

This invention relates generally to neurodegenerative diseases and conditions (e.g., Alzheimer's disease) characterized with higher than normal brain blood ammonia levels and/or higher than normal amounts of circulatory pro-inflammatory cytokines secreted by harmful gut bacteria. This invention further relates to methods and compositions for treating such neurodegenerative diseases and conditions with pharmaceutical compositions capable of reducing blood ammonia levels and/or reducing levels of circulatory pro-inflammatory cytokines secreted by harmful gut bacteria.

### FIELD OF THE INVENTION

This invention relates generally to neurodegenerative diseases and conditions (e.g., Alzheimer's disease) characterized with higher than normal brain blood ammonia levels and/or higher than normal amounts of circulatory pro-inflammatory cytokines secreted by harmful gut bacteria. This invention further relates to methods and compositions for treating such neurodegenerative diseases and conditions with pharmaceutical compositions capable of reducing blood ammonia levels and/or reducing levels of circulatory pro-inflammatory cytokines secreted by harmful gut bacteria.

### BACKGROUND OF THE INVENTION

There is an urgent need to develop novel therapies for neurodegenerative diseases and conditions such as Alzheimer's disease (AD). 10% of persons over age 65 and up to 50% over age 85 have dementia, with over 30 million people affected worldwide. The Food and Drug Administration (FDA) has approved cholinesterase inhibitors (e.g., Aricept, Exelon, Razadyne), memantine (Namenda), and a drug combining a cholinesterase inhibitor and memantine (Namzaric) to treat the cognitive symptoms of neurodegenerative disorders (e.g., AD). These medications, however, cannot cure neurodegenerative disorders or stop related progression.

As such, improved methods for treating neurodegenerative disorders (e.g., AD) are needed.

The present invention addresses this need.

### SUMMARY

Alzheimer's disease (AD) is the most frequent cause of dementia characterized by a progressive decline in cognitive function associated with the formation of amyloid beta (Aβ) plaques and neurofibrillary tangles. There has been much speculation for decades regarding the cause of Alzheimer's Disease. A first theory posits that toxic brain concentrations of ammonia play a role in the AD pathophysiology (see, Seiler N. Neurochemical Res. 1993;18:235-245). There have, however, been no published trials of using ammonia lowering medications for the treatment of AD. An additional and more recent theory proposes that there is a symbiotic relationship between gut microbiota and brain homeostasis and that this relationship is disrupted in patients with AD, possibly due to an imbalance in the ratio of pro-inflammatory/anti-inflammatory gut microbiota (see, De-Paula, V et al. Pharmacological Research 2018;136:29-34; Cattaneo A, et al. Neurobiology of Aging 2017;49:60-68). Alterations in the gut microbiota composition induce increased permeability of the gut barrier and immune activation leading to systemic inflammation, which in turn may impair the blood-brain barrier and promote neuroinflammation, neural injury, and ultimately neurodegeneration.

Rifaximin is a virtually non-absorbed antibiotic with the unique properties of lowering blood ammonia levels and altering gut flora. It is hypothesized that administration of rifaximin to patients suffering from neurodegenerative disorders (e.g., AD, Parkinson's disease, Huntington's disease, amyotrophic lateral sclerosis, motor neuron disease) will result in improved cognition and function in such patients by lowering blood ammonia and/or lowering circulatory pro-inflammatory cytokines secreted by harmful gut bacteria. Indeed, it is hypothesized that administration of rifaximin to such patients will result in an increase in small bowel glutaminase levels, an alteration in serum amyloid-beta 42 (Aβ-42) levels, a reduction in total-tau levels, an alteration in neurofilament light protein marker levels, a reduction in serum pro-inflammatory markers (e.g., IL-2, IL-4, IL-5, IL-6, IL-8, IL-10, IL-13, tumor necrosis factor alpha), a reduction in serum ammonia levels, and a reduction in pro-inflammatory cytokines secreted by harmful gut bacteria.

For purposes of establishing these hypotheses experiments will be conducted that measure blood ammonia levels and pro-inflammatory and anti-inflammatory markers in the blood and analyzing gut microbiota in patients with AD before and after three months of rifaximin therapy. Patients exhibiting measurable improvement in cognition and function will be further analyzed to determine if such improvement correlates with a lower blood ammonia level and/or a shift towards anti-inflammatory species in the gut and a similar shift in the blood cytokine panel to favoring anti-inflammatory compounds. It is anticipated that administration of rifaximin to patients suffering from neurodegenerative disorders (e.g., AD) will improve cognition and function in such patients by lowering blood ammonia levels and/or lowering the amount of circulatory pro-inflammatory cytokines secreted by harmful gut bacteria. In addition, it is anticipated that such rifaximin treatment will further result in an increase in small bowel glutaminase levels, an alteration in serum amyloid-beta 42 (Aβ-42) levels, a reduction in total-tau levels, an alteration in neurofilament light protein marker levels, and a reduction in serum pro-inflammatory markers (e.g., IL-2, IL-4, IL-5, IL-6, IL-8, IL-10, IL-13, tumor necrosis factor alpha).

Accordingly, the present invention provides methods for treating, preventing and/or ameliorating symptoms of neurodegenerative disorders (e.g., AD, Parkinson's disease, Huntington's disease, amyotrophic lateral sclerosis, motor neuron disease) through one or more of lowering blood ammonia, increasing small bowel glutaminase levels, lowering circulatory pro-inflammatory cytokines secreted by harmful gut bacteria (e.g., IL-2, IL-4, IL-5, IL-6, IL-8, IL-10, IL-13, tumor necrosis factor alpha), altering serum amyloid-beta 42 levels, lowering total-tau levels, altering neurofilament light protein markers, and lowering gut microbiota levels. In some embodiments, altering the gut microbiota affects pathology related to AD via amyloid and tau processing. In some embodiments, altering the gut microbiota affects cognition or behavior related to AD via modulating metabolism of neurotransmitters/neuropeptides such as acetylcholine or serotonin or norepinephrine.

Such methods are not limited to use of a particular agent capable of lowering blood ammonia, increasing small bowel glutaminase levels, lowering circulatory pro-inflammatory cytokines secreted by harmful gut bacteria (e.g., IL-2, IL-4, IL-5, IL-6, IL-8, IL-10, IL-13, tumor necrosis factor alpha), altering serum amyloid-beta 42 levels, lowering total-tau levels, altering neurofilament light protein markers, and/or lowering gut microbiota levels. In some embodiments, the agent is a pharmaceutical composition comprising a non-systematically absorbed antibiotic. In other embodiments, the agent is capable of (a) altering fecal flora in the subject by blocking bacterial RNA synthesis and (b) increasing small bowel glutaminase. In some embodiments, the agent is a pharmaceutical composition comprising a therapeutically effective amount of rifaximin or any derivatives, salts and esters thereof. In some embodiments, the agent is a pharmaceutical composition comprising a therapeutically effective amount of one or more of sodium benzoate, sodium phenylacetate, glycerol phenylbutyrate, ornithine phenylacetate, AST-120 (spherical carbon adsorbent), and polyethylene glycol, or any derivatives, salts and esters thereof.

In certain embodiments, the present invention provides a method for lowering blood and brain ammonia levels in a subject suffering from a neurodegenerative disorder (e.g., AD, Parkinson's disease, Huntington's disease, amyotrophic lateral sclerosis, motor neuron disease) comprising, consisting of, or consisting essentially of administering to the subject a therapeutically effective amount of an agent capable of lowering blood ammonia, increasing small bowel glutaminase levels, lowering circulatory pro-inflammatory cytokines secreted by harmful gut bacteria (e.g., IL-2, IL-4, IL-5, IL-6, IL-8, IL-10, IL-13, tumor necrosis factor alpha), altering serum amyloid-beta 42 levels, lowering total-tau levels, altering neurofilament light protein markers, and/or lowering gut microbiota levels (e.g., rifaximin, sodium benzoate, sodium phenylacetate, glycerol phenylbutyrate, ornithine phenylacetate, AST-120 (spherical carbon adsorbent), and polyethylene glycol, or any derivatives, salts and esters thereof).

In certain embodiments, the present invention provides a method for lowering circulatory pro-inflammatory cytokines secreted by harmful gut bacteria (e.g., IL-2, IL-4, IL-5, IL-6, IL-8, IL-10, IL-13, tumor necrosis factor alpha) in a subject suffering from a neurodegenerative disorder (e.g., AD, Parkinson's disease, Huntington's disease, amyotrophic lateral sclerosis, motor neuron disease) comprising, consisting of, or consisting essentially of administering to the subject a therapeutically effective amount of an agent capable of lowering blood ammonia, increasing small bowel glutaminase levels, lowering circulatory pro-inflammatory cytokines secreted by harmful gut bacteria (e.g., IL-2, IL-4, IL-5, IL-6, IL-8, IL-10, IL-13, tumor necrosis factor alpha), altering serum amyloid-beta 42 levels, lowering total-tau levels, altering neurofilament light protein markers, and/or lowering gut microbiota levels (e.g., rifaximin, sodium benzoate, sodium phenylacetate, glycerol phenylbutyrate, ornithine phenylacetate, AST-120 (spherical carbon adsorbent), and polyethylene glycol, or any derivatives, salts and esters thereof).

In certain embodiments, the present invention provides a method for increasing small bowel glutaminase levels in a subject suffering from a neurodegenerative disorder (e.g., AD, Parkinson's disease, Huntington's disease, amyotrophic lateral sclerosis, motor neuron disease) comprising, consisting of, or consisting essentially of administering to the subject a therapeutically effective amount of an agent capable of lowering blood ammonia, increasing small bowel glutaminase levels, lowering circulatory pro-inflammatory cytokines secreted by harmful gut bacteria (e.g., IL-2, IL-4, IL-5, IL-6, IL-8, IL-10, IL-13, tumor necrosis factor alpha), altering serum amyloid-beta 42 levels, lowering total-tau levels, altering neurofilament light protein markers, and/or lowering gut microbiota levels (e.g., rifaximin, sodium benzoate, sodium phenylacetate, glycerol phenylbutyrate, ornithine phenylacetate, AST-120 (spherical carbon adsorbent), and polyethylene glycol, or any derivatives, salts and esters thereof).

In certain embodiments, the present invention provides a method for altering serum amyloid-beta 42 levels in a subject suffering from a neurodegenerative disorder (e.g., AD, Parkinson's disease, Huntington's disease, amyotrophic lateral sclerosis, motor neuron disease) comprising, consisting of, or consisting essentially of administering to the subject a therapeutically effective amount of an agent capable of lowering blood ammonia, increasing small bowel glutaminase levels, lowering circulatory pro-inflammatory cytokines secreted by harmful gut bacteria (e.g., IL-2, IL-4, IL-5, IL-6, IL-8, IL-10, IL-13, tumor necrosis factor alpha), altering serum amyloid-beta 42 levels, lowering total-tau levels, altering neurofilament light protein markers, and/or lowering gut microbiota levels (e.g., rifaximin, sodium benzoate, sodium phenylacetate, glycerol phenylbutyrate, ornithine phenylacetate, AST-120 (spherical carbon adsorbent), and polyethylene glycol, or any derivatives, salts and esters thereof),In certain embodiments, the present invention provides a method for lowering total tau levels in a subject suffering from a neurodegenerative disorder (e.g., AD, Parkinson's disease, Huntington's disease, amyotrophic lateral sclerosis, motor neuron disease) comprising, consisting of, or consisting essentially of administering to the subject a therapeutically effective amount of rifaximin or any derivatives, salts and esters thereof.

In certain embodiments, the present invention provides a method for altering neurofilament light protein marker levels in a subject suffering from a neurodegenerative disorder (e.g., AD, Parkinson's disease, Huntington's disease, amyotrophic lateral sclerosis, motor neuron disease) comprising, consisting of, or consisting essentially of administering to the subject a therapeutically effective amount of an agent capable of lowering blood ammonia, increasing small bowel glutaminase levels, lowering circulatory pro-inflammatory cytokines secreted by harmful gut bacteria (e.g., IL-2, IL-4, IL-5, IL-6, IL-8, IL-10, IL-13, tumor necrosis factor alpha), altering serum amyloid-beta 42 levels, lowering total-tau levels, altering neurofilament light protein markers, and/or lowering gut microbiota levels (e.g., rifaximin, sodium benzoate, sodium phenylacetate, glycerol phenylbutyrate, ornithine phenylacetate, AST-120 (spherical carbon adsorbent), and polyethylene glycol, or any derivatives, salts and esters thereof).

In certain embodiments, the present invention provides a method for lowering gut microbiota levels in a subject suffering from a neurodegenerative disorder (e.g., AD, Parkinson's disease, Huntington's disease, amyotrophic lateral sclerosis, motor neuron disease) comprising, consisting of, or consisting essentially of administering to the subject a therapeutically effective amount of an agent capable of lowering blood ammonia, increasing small bowel glutaminase levels, lowering circulatory pro-inflammatory cytokines secreted by harmful gut bacteria (e.g., IL-2, IL-4, IL-5, IL-6, IL-8, IL-10, IL-13, tumor necrosis factor alpha), altering serum amyloid-beta 42 levels, lowering total-tau levels, altering neurofilament light protein markers, and/or lowering gut microbiota levels (e.g., rifaximin, sodium benzoate, sodium phenylacetate, glycerol phenylbutyrate, ornithine phenylacetate, AST-120 (spherical carbon adsorbent), and polyethylene glycol, or any derivatives, salts and esters thereof).

Another aspect of the present invention provides a method of preventing the onset of a neurodegenerative disorder (e.g., AD, Parkinson's disease, Huntington's disease, amyotrophic lateral sclerosis, motor neuron disease) comprising, consisting of, or consisting essentially of administering to a subject a therapeutically effective amount of a compound capable of one or more of lowering blood ammonia, increasing small bowel glutaminase levels, lowering circulatory pro-inflammatory cytokines secreted by harmful gut bacteria (e.g., IL-2, IL-4, IL-5, IL-6, IL-8, IL-10, IL-13, tumor necrosis factor alpha), altering serum amyloid-beta 42 levels, lowering total-tau levels, altering neurofilament light protein markers, and lowering gut microbiota levels in the subject such that the onset of the neurodegenerative disorder is prevented. In some embodiments, the compound is one or more of rifaximin, sodium benzoate, sodium phenylacetate, glycerol phenylbutyrate, ornithine phenylacetate, AST-120 (spherical carbon adsorbent), and polyethylene glycol, or any derivatives, salts and esters thereof.

In certain embodiments, the present invention is also directed to methods of screening agents for preventing and/or ameliorating symptoms of neurodegenerative disorders (e.g., AD, Parkinson's disease, Huntington's disease, amyotrophic lateral sclerosis, motor neuron disease) in a mammal. In some embodiments, such methods comprise administering to a patient suffering from, for example, AD a candidate agent, and comparing the result of such administration to an established norm in terms of ability to improve cognitive function, lower blood ammonia, increase small bowel glutaminase levels, lower circulatory pro-inflammatory cytokines secreted by harmful gut bacteria (e.g., IL-2, IL-4, IL-5, IL-6, IL-8, IL-10, IL-13, tumor necrosis factor alpha), altera serum amyloid-beta 42 levels, lower total-tau levels, alter neurofilament light protein markers, and/or lower gut microbiota levels.

### DEFINITIONS

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. Embodiments recited as “including,” “comprising” or “having” certain elements are also contemplated as “consisting essentially of and “consisting of those certain elements.

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

Unless otherwise defined, all technical terms used herein have the same meaning as commonly understood by one of ordinary skill in the art to which this disclosure belongs.

### DETAILED DESCRIPTION

Ammonia is a substrate, as well as a product, of 16 different enzymatic reactions in the brain. In a healthy brain, ammonia homeostasis is maintained by its combining with glutamate to form non-toxic glutamine via the glutamine synthetase pathway. In the neurodegenerative disease state, high brain ammonia levels adversely affect membrane potential, mitochondrial function, astrocyte morphology, energy metabolism, mRNA and protein expression, brain pH, calcium signaling and other cellular functions in the brain (see, Bosoi, C R et al. Metabolic Brain Disease 2009;24:95-102).

The possibility that ammonia is at least partly responsible for the pathologic changes seen in the AD brain was first proposed in 1993, when it was noted that some of the changes in the AD brain can also be seen in the brains of hyperammonemic patients (see, Seiler N. Neurochemical Res. 1993;18:235-245). The clinical presentation of a patient with high ammonia levels caused by congenital portal systemic shunting or an inborn error of metabolism may in fact mimic that of dementia (see, Miyata K et al. Intern Med 2009;48:321-4). In addition, brain PET CT using 13N ammonia showed high brain ¹³N levels, trapped in glutamine, in the brain of patients with hepatic encephalopathy, correlating with high peripheral arterial blood ammonia levels (see, Keiding, S et al. Hepatology 2006;43:42-50).

Ammonia as a causative factor of AD can be difficult to prove; ammonia levels cannot be quantified at autopsy because of rapid post mortem formation of ammonia. However, in-vivo studies have shown evidence of elevated ammonia levels in the AD brain. Arteriovenous sampling in early stage normoammonemic AD patients demonstrated a net release of ammonia (−25.6 micrograms/100 g×min) from the brain compared to net uptake of ammonia (+7.2 micrograms/100 g×min) by the brain of young control subjects, pointing toward an endogenous source for the ammonia (see, Hoyer, S et al. Neuroscience Letters 1990;117:358-362). This efflux of ammonia from the AD brain is most likely due to low levels of glutamine synthetase found in the AD brain (see, Le Prince, G et al. Neurochemical Research 1995;7:859-862), causing high levels of this endogenous ammonia to build up in the extracellular fluid and diffuse across the blood brain barrier into the venous effluent. Low glutamine synthetase levels have been found to correlate with increased density of amyloid deposits in the AD brain (see, Le Prince, Get al. Neurochemical Research 1995;7:859-862).

The brain also receives exogenous ammonia, of which the gut is a major source. Fecal bacteria produce ammonia by fermenting protein. The aging colon contains a greater percentage of protein fermenting bacteria than is seen in the colon of younger patients (see, Woodmansey E J. Journal of Applied Microbiology 2007;102:1178-1186; Andrieux C et al Scand J Gastroenterol 2002;37:792-8). The aging colon also has a longer fecal dwell time than that of younger patients, allowing higher levels of ammonia to build up before fecal evacuation (see, Woodmansey E J. Journal of Applied Microbiology 2007;102:1178-1186; Andrieux C et al Scand J Gastroenterol 2002;37:792-8). Gut ammonia is absorbed into the portal venous system and detoxified in the liver to form uric acid, which is then excreted in the kidneys. The uric acid cycle of the aging liver is less efficient at detoxifying ammonia (see, Marchesini G et al. Ageing 1990;19:4-10) allowing higher levels of ammonia to reach the systemic circulation. One study reported significantly elevated post prandial blood ammonia levels in AD patients when compared with age-matched controls (see, Fisman M et al. Am J Psychiatry 1985;142:71-73).

The blood brain barrier plays an important role in regulating brain ammonia metabolism (see, Hawkins R A. Am J Clin Nutr. 2009;90:867S-74S). 20-50% of blood ammonia passes the blood brain barrier and is converted into glutamine. Glutamine and glutamate are pumped from the extracellular fluid into the endothelial cells, where glutamine is partially metabolized to ammonia and glutamate. Glutamine and ammonia then diffuse into the blood.

Breakdown of the blood brain barrier in the aging patient begins in the hippocampus, the area critical for learning and memory, and this may contribute to cognitive decline (see, Erdo F et al. Journal of Cerebral Blood Flow and Metabolism 2017;37:4-24). Gadolinium enhanced Brain MRI has shown that the blood brain barrier is more permeable in the aging patient when compared with younger patients (see, Montagne, A et al. Blood-Brain Barrier Breakdown in the Aging Human Hippocampus. Neuron 2015;85:296-302). The blood brain barrier in the AD patient is also more permeable than that of non-cognitively-impaired age matched controls (see, Montagne, A et al. Blood-Brain Barrier Breakdown in the Aging Human Hippocampus. Neuron 2015;85:296-302). High concentrations of Gadolinium are first seen in the hippocampus, the site responsible for memory and learning, both of which are clinically impaired in AD patients.

As such, it appears that the blood brain barrier allows greater amounts of ammonia into the AD brain, triggering or worsening pre-existent changes.

Experiments will be conducted with the purpose of lowering blood and brain ammonia levels in AD patients by targeting the colon. This will be accomplished by administering a virtually non-systemically absorbed antibiotic, rifaximin, which is FDA approved for hepatic encephalopathy. Rifaximin lowers blood ammonia by altering fecal flora by blocking bacterial RNA synthesis and also by increasing small bowel glutaminase (see, Garcovich, M. World J Gastroenterol 2012;18:6693-6700; Kang D J et al. Clinical and Translational Gastroenterology 2016;7:e187).

The gut harbors 95% of the total human microbiome and is made up of more than 5,000 taxa. A diverse and stable gut microbiota promotes health in humans. Gut species remain relatively constant throughout adulthood until the seventh decade, when it becomes less diverse, harboring higher numbers of Proteobacteria and lower numbers of Bifidobacteria. This change most likely accounts for chronic inflammatory disorders seen in the elderly. An imbalance in the gut bacterial species can weaken the intestinal barrier and create system wide inflammation via gut lymphoid tissue which comprises 70%-80% of the immune system. Blood brain barrier permeability is also altered. The gut bacteria secrete pro-inflammatory compounds and neuroactive molecules that include serotonin, gamma-aminobutyric acid (GABA), catecholamines and acetylcholine which cross the blood brain barrier and cause brain inflammation and brain dysfunction (see, Sochocka M, et al. Molecular Neurobiology 2018; pp 1-11).

A growing number of pro-inflammatory compounds secreted by gut bacteria and seen in patients with AD are being discovered. These include interleukin (IL)—6, tumor necrosis—alpha and the inflammasome complex (NLRP3). A recent study revealed higher numbers of Escherichia and Shigella in the gut of amyloid positive AD patients when compared with healthy controls and this correlated with higher levels of circulating pro-inflammatory cytokines. The AD patients also had lower numbers of gut Eubacterium rectale and this correlated with lower levels of circulating anti-inflammatory compounds than seen in controls (see, Cattaneo A, et al. Neurobiology of Aging 2017;49:60-68). Other researchers found that a preponderance of gut Bacteroides and Blautia and reduced numbers of SMB53 and Dialister correlated with elevated levels of brain amyloid CSF biomarkers (see, Vogte N M et al. Sci Rep. 2017;7:13537). Interestingly, Clostridium tyrobutyricum and Bacteroides thetaiotaomicron, have been shown to actually increase the integrity of the blood brain barrier by enhancing expression of tight junction proteins and helping to maintain brain homeostasis (see, Braniste V et al. Sci Transl Med 2014;6:263).

Recent evidence also points to a possibly more direct influence of gut bacteria on the brain. Terminal branches of the vagus nerve travel in close proximity to the intestinal barrier. Neurotransmitters and neuropeptides secreted by bacteria in the gut have been shown to activate vagal nerve ascending fibers, influencing brain function in mice. These effects ceased after vagotomy was performed (see, Holzer P et al. Adv Exp Med Biol 817:195-219; Bravo JA et al. PNAS 108:16050-16055). For purposes of establishing these hypotheses experiments will be conducted that measure blood ammonia levels and pro-inflammatory and anti-inflammatory markers in the blood and analyzing gut microbiota in patients with AD before and after three months of rifaximin therapy. Patients exhibiting measurable improvement in cognition and function will be further analyzed to determine if such improvement correlates with a lower blood ammonia level and/or a shift towards anti-inflammatory species in the gut and a similar shift in the blood cytokine panel to favoring anti-inflammatory compounds. It is anticipated that administration of rifaximin to patients suffering from neurodegenerative disorders (e.g., AD) will improve cognition and function in such patients by lowering blood ammonia levels and/or lowering the amount of circulatory pro-inflammatory cytokines secreted by harmful gut bacteria. In addition, it is anticipated that such rifaximin treatment will further result in an increase in small bowel glutaminase levels, an alteration in serum amyloid-beta 42 (Aβ-42) levels, a reduction in total-tau levels, an alteration in neurofilament light protein marker levels, and a reduction in serum pro-inflammatory markers (e.g., IL-2, IL-4, IL-5, IL-6, IL-8, IL-10, IL-13, tumor necrosis factor alpha).

Neurofilament light (NF-L) is a 68 kDa cytoskeletal intermediate filament protein that is expressed in neurons (see, Mattson N et al. JAMA Neurol. 2017;74(5):557-566; Rissin, D et al. Nature Biotechnology 28, 595-599 (2010)). Neurofilaments can be released in significant quantity following axonal damage or neuronal degeneration. NF-L has been shown to associate with traumatic brain injury, multiple sclerosis, frontotemporal dementia and other neurodegenerative diseases.

Tau is a microtubule-stabilizing protein primarily localized in central nervous system neurons, but is also expressed at low levels in astrocytes and oligodendrocytes (see, Dage J L et al. Alzheimer's & Dementia, 12, 1226-1234, 2016). Potential movement of elevated CSF tau across the blood-brain barrier presents a possibility that measurements of tau in blood could provide a convenient peripheral window into brain/CSF status. Recent reports using digital immunoassay technology have shown elevation in peripheral tau associated with hypoxic brain injury, concussed hockey players, and repetitive minimal head injury.

Amyloid beta 42 is a 42 amino acid proteolytic product from the amyloid precursor protein that has gained considerable attention as a biomarker correlating with cognitive disorders (see, Janelidze S et al. Nature Scientific Reports, 6, 26801, 2016). Amyloid beta (Aβ) peptides (including the shorter A1338 and A1340 isoforms) are produced by many cell types in the body but the expression is particularly high in the brain. Accumulation of Aβ occurs in aging and in the neurodegenerative process.

Accordingly, the present invention provides methods for preventing and/or ameliorating symptoms of neurodegenerative disorders (e.g., AD, Parkinson's disease, Huntington's disease, amyotrophic lateral sclerosis, motor neuron disease) through one or more of lowering blood ammonia, increasing small bowel glutaminase levels, lowering circulatory pro-inflammatory cytokines secreted by harmful gut bacteria (e.g., IL-2, IL-4, IL-5, IL-6, IL-8, IL-10, IL-13, tumor necrosis factor alpha), altering serum amyloid-beta 42 levels, lowering total-tau levels, altering neurofilament light protein markers, and lowering gut microbiota levels. In some embodiments, altering the gut microbiota affects pathology related to AD via amyloid and tau processing. In some embodiments, altering the gut microbiota affects cognition or behavior related to AD via modulating metabolism of neurotransmitters/neuropeptides such as acetylcholine or serotonin or norepinephrine.

Such methods are not limited to use of a particular agent capable of lowering blood ammonia, increasing small bowel glutaminase levels, lowering circulatory pro-inflammatory cytokines secreted by harmful gut bacteria (e.g., IL-2, IL-4, IL-5, IL-6, IL-8, IL-10, IL-13, tumor necrosis factor alpha), altering serum amyloid-beta 42 levels, lowering total-tau levels, altering neurofilament light protein markers, and/or lowering gut microbiota levels. Indeed, such agents can include, but are not limited to, one or more of: small molecules, inhibitory nucleic acids, antibodies, and inhibitory peptides.

In some embodiments, the agent is a pharmaceutical composition comprising a non-systematically absorbed antibiotic. In other embodiments, the agent is capable of (a) altering fecal flora in the subject by blocking bacterial RNA synthesis and (b) increasing small bowel glutaminase. In some embodiments, the agent is a pharmaceutical composition comprising a therapeutically effective amount of rifaximin or any derivatives, salts and esters thereof.

In some embodiments, the agent is a pharmaceutical composition comprising a therapeutically effective amount of one or more of sodium benzoate, sodium phenylacetate, glycerol phenylbutyrate, ornithine phenylacetate, AST-120 (spherical carbon adsorbent), and polyethylene glycol, or any derivatives, salts and esters thereof.

In certain embodiments, the present invention provides a method for lowering blood and brain ammonia levels in a subject suffering from a neurodegenerative disorder (e.g., AD, Parkinson's disease, Huntington's disease, amyotrophic lateral sclerosis, motor neuron disease) comprising, consisting of, or consisting essentially of administering to the subject a therapeutically effective amount of an agent capable of lowering blood ammonia, increasing small bowel glutaminase levels, lowering circulatory pro-inflammatory cytokines secreted by harmful gut bacteria (e.g., IL-2, IL-4, IL-5, IL-6, IL-8, IL-10, IL-13, tumor necrosis factor alpha), altering serum amyloid-beta 42 levels, lowering total-tau levels, altering neurofilament light protein markers, and/or lowering gut microbiota levels (e.g., rifaximin, sodium benzoate, sodium phenylacetate, glycerol phenylbutyrate, ornithine phenylacetate, AST-120 (spherical carbon adsorbent), and polyethylene glycol, or any derivatives, salts and esters thereof).

In certain embodiments, the present invention provides a method for lowering circulatory pro-inflammatory cytokines secreted by harmful gut bacteria (e.g., IL-2, IL-4, IL-5, IL-6, IL-8, IL-10, IL-13, tumor necrosis factor alpha) in a subject suffering from a neurodegenerative disorder (e.g., AD, Parkinson's disease, Huntington's disease, amyotrophic lateral sclerosis, motor neuron disease) comprising, consisting of, or consisting essentially of administering to the subject a therapeutically effective amount of an agent capable of lowering blood ammonia, increasing small bowel glutaminase levels, lowering circulatory pro-inflammatory cytokines secreted by harmful gut bacteria (e.g., IL-2, IL-4, IL-5, IL-6, IL-8, IL-10, IL-13, tumor necrosis factor alpha), altering serum amyloid-beta 42 levels, lowering total-tau levels, altering neurofilament light protein markers, and/or lowering gut microbiota levels (e.g., rifaximin, sodium benzoate, sodium phenylacetate, glycerol phenylbutyrate, ornithine phenylacetate, AST-120 (spherical carbon adsorbent), and polyethylene glycol, or any derivatives, salts and esters thereof).

In certain embodiments, the present invention provides a method for increasing small bowel glutaminase levels in a subject suffering from a neurodegenerative disorder (e.g., AD, Parkinson's disease, Huntington's disease, amyotrophic lateral sclerosis, motor neuron disease) comprising, consisting of, or consisting essentially of administering to the subject a therapeutically effective amount of an agent capable of lowering blood ammonia, increasing small bowel glutaminase levels, lowering circulatory pro-inflammatory cytokines secreted by harmful gut bacteria (e.g., IL-2, IL-4, IL-5, IL-6, IL-8, IL-10, IL-13, tumor necrosis factor alpha), altering serum amyloid-beta 42 levels, lowering total-tau levels, altering neurofilament light protein markers, and/or lowering gut microbiota levels (e.g., rifaximin, sodium benzoate, sodium phenylacetate, glycerol phenylbutyrate, ornithine phenylacetate, AST-120 (spherical carbon adsorbent), and polyethylene glycol, or any derivatives, salts and esters thereof).

In certain embodiments, the present invention provides a method for altering serum amyloid-beta 42 levels in a subject suffering from a neurodegenerative disorder (e.g., AD, Parkinson's disease, Huntington's disease, amyotrophic lateral sclerosis, motor neuron disease) comprising, consisting of, or consisting essentially of administering to the subject a therapeutically effective amount of an agent capable of lowering blood ammonia, increasing small bowel glutaminase levels, lowering circulatory pro-inflammatory cytokines secreted by harmful gut bacteria (e.g., IL-2, IL-4, IL-5, IL-6, IL-8, IL-10, IL-13, tumor necrosis factor alpha), altering serum amyloid-beta 42 levels, lowering total-tau levels, altering neurofilament light protein markers, and/or lowering gut microbiota levels (e.g., rifaximin, sodium benzoate, sodium phenylacetate, glycerol phenylbutyrate, ornithine phenylacetate, AST-120 (spherical carbon adsorbent), and polyethylene glycol, or any derivatives, salts and esters thereof).

In certain embodiments, the present invention provides a method for lowering total tau levels in a subject suffering from a neurodegenerative disorder (e.g., AD, Parkinson's disease, Huntington's disease, amyotrophic lateral sclerosis, motor neuron disease) comprising, consisting of, or consisting essentially of administering to the subject a therapeutically effective amount of an agent capable of lowering blood ammonia, increasing small bowel glutaminase levels, lowering circulatory pro-inflammatory cytokines secreted by harmful gut bacteria (e.g., IL-2, IL-4, IL-5, IL-6, IL-8, IL-10, IL-13, tumor necrosis factor alpha), altering serum amyloid-beta 42 levels, lowering total-tau levels, altering neurofilament light protein markers, and/or lowering gut microbiota levels (e.g., rifaximin, sodium benzoate, sodium phenylacetate, glycerol phenylbutyrate, ornithine phenylacetate, AST-120 (spherical carbon adsorbent), and polyethylene glycol, or any derivatives, salts and esters thereof).

In certain embodiments, the present invention provides a method for altering neurofilament light protein marker levels in a subject suffering from a neurodegenerative disorder (e.g., AD, Parkinson's disease, Huntington's disease, amyotrophic lateral sclerosis, motor neuron disease) comprising, consisting of, or consisting essentially of administering to the subject a therapeutically effective amount of an agent capable of lowering blood ammonia, increasing small bowel glutaminase levels, lowering circulatory pro-inflammatory cytokines secreted by harmful gut bacteria (e.g., IL-2, IL-4, IL-5, IL-6, IL-8, IL-10, IL-13, tumor necrosis factor alpha), altering serum amyloid-beta 42 levels, lowering total-tau levels, altering neurofilament light protein markers, and/or lowering gut microbiota levels (e.g., rifaximin, sodium benzoate, sodium phenylacetate, glycerol phenylbutyrate, ornithine phenylacetate, AST-120 (spherical carbon adsorbent), and polyethylene glycol, or any derivatives, salts and esters thereof).

In certain embodiments, the present invention provides a method for lowering gut microbiota levels in a subject suffering from a neurodegenerative disorder (e.g., AD, Parkinson's disease, Huntington's disease, amyotrophic lateral sclerosis, motor neuron disease) comprising, consisting of, or consisting essentially of administering to the subject a therapeutically effective amount of an agent capable of lowering blood ammonia, increasing small bowel glutaminase levels, lowering circulatory pro-inflammatory cytokines secreted by harmful gut bacteria (e.g., IL-2, IL-4, IL-5, IL-6, IL-8, IL-10, IL-13, tumor necrosis factor alpha), altering serum amyloid-beta 42 levels, lowering total-tau levels, altering neurofilament light protein markers, and/or lowering gut microbiota levels (e.g., rifaximin, sodium benzoate, sodium phenylacetate, glycerol phenylbutyrate, ornithine phenylacetate, AST-120 (spherical carbon adsorbent), and polyethylene glycol, or any derivatives, salts and esters thereof).

The methods and compositions of the present invention are useful in treating mammals. Such mammals include humans as well as non-human mammals. Non-human mammals include, for example, companion animals such as dogs and cats, agricultural animals such live stock including cows, horses and the like, and exotic animals, such as zoo animals.

Treatment can include administration of an effective amount of one or more of an agent capable of lowering blood ammonia, increasing small bowel glutaminase levels, lowering circulatory pro-inflammatory cytokines secreted by harmful gut bacteria (e.g., IL-2, IL-4, IL-5, IL-6, IL-8, IL-10, IL-13, tumor necrosis factor alpha), altering serum amyloid-beta 42 levels, lowering total-tau levels, altering neurofilament light protein markers, and/or lowering gut microbiota levels.

Administration can be by any suitable route of administration including buccal, dental, endocervical, intramuscular, inhalation, intracranial, intralymphatic, intramuscular, intraocular, intraperitoneal, intrapleural, intrathecal, intratracheal, intrauterine, intravascular, intravenous, intravesical, intranasal, ophthalmic, oral, otic, biliary perfusion, cardiac perfusion, priodontal, rectal, spinal subcutaneous, sublingual, topical, intravaginal, transermal, ureteral, or urethral. Dosage forms can be aerosol including metered aerosol, chewable bar, capsule, capsule containing coated pellets, capsule containing delayed release pellets, capsule containing extended release pellets, concentrate, cream, augmented cream, suppository cream, disc, dressing, elixer, emulsion, enema, extended release fiber, extended release film, gas, gel, metered gel, granule, delayed release granule, effervescent granule, chewing gum, implant, inhalant, injectable, injectable lipid complex, injectable liposomes, insert, extended release insert, intrauterine device, jelly, liquid, extended release liquid, lotion, augmented lotion, shampoo lotion, oil, ointment, augmented ointment, paste, pastille, pellet, powder, extended release powder, metered powder, ring, shampoo, soap solution, solution for slush, solution/drops, concentrate solution, gel forming solution/drops, sponge, spray, metered spray, suppository, suspension, suspension/drops, extended release suspension, swab, syrup, tablet, chewable tablet, tablet containing coated particles, delayed release tablet, dispersible tablet, effervescent tablet, extended release tablet, orally disintegrating tablet, tampon, tape or troche/lozenge.

Intraocular administration can include administration by injection including intravitreal injection, by eyedrops and by trans-scleral delivery.

Administration can also be by inclusion in the diet of the mammal such as in a functional food for humans or companion animals.

It is also contemplated that certain formulations containing the compositions capable of lowering blood ammonia, increasing small bowel glutaminase levels, lowering circulatory pro-inflammatory cytokines secreted by harmful gut bacteria (e.g., IL-2, IL-4, IL-5, IL-6, IL-8, IL-10, IL-13, tumor necrosis factor alpha), altering serum amyloid-beta 42 levels, lowering total-tau levels, altering neurofilament light protein markers, and/or lowering gut microbiota levels are to be administered orally. Such formulations are preferably encapsulated and formulated with suitable carriers in solid dosage forms. Some examples of suitable carriers, excipients, and diluents include lactose, dextrose, sucrose, sorbitol, mannitol, starches, gum acacia, calcium phosphate, alginates, calcium silicate, microcrystalline cellulose, polyvinylpyrrolidone, cellulose, gelatin, syrup, methylcellulose, methyl- and propylhydroxybenzoates, talc, magnesium, stearate, water, mineral oil, and the like. The formulations can additionally include lubricating agents, wetting agents, emulsifying and suspending agents, preserving agents, sweetening agents or flavoring agents. The compositions may be formulated such as to provide rapid, sustained, or delayed release of the active ingredients after administration to the patient by employing procedures well known in the art. The formulations can also contain substances that diminish proteolytic degradation and promote absorption such as, for example, surface-active agents.

The specific dose can be calculated according to the approximate body weight or body surface area of the patient or the volume of body space to be occupied. The dose will also depend upon the particular route of administration selected. Further refinement of the calculations necessary to determine the appropriate dosage for treatment is routinely made by those of ordinary skill in the art. Such calculations can be made without undue experimentation by one skilled in the art in light of the activity in assay preparations such as has been described elsewhere for certain compounds (see for example, Howitz et al., Nature 425:191-196, 2003 and supplementary information that accompanies the paper). Exact dosages can be determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

The present invention also provides kits comprising an agent (e.g., rifaximin) capable of lowering blood ammonia, increasing small bowel glutaminase levels, lowering circulatory pro-inflammatory cytokines secreted by harmful gut bacteria (e.g., IL-2, IL-4, IL-5, IL-6, IL-8, IL-10, IL-13, tumor necrosis factor alpha), altering serum amyloid-beta 42 levels, lowering total-tau levels, altering neurofilament light protein markers, and/or lowering gut microbiota levels and instructions for administering the agent to an animal (e.g., a human patient suffering from a neurodegenerative disorder (e.g., AD)). The kits may optionally contain other therapeutic agents.

### EXPERIMENTAL

The following examples are provided to demonstrate and further illustrate certain preferred embodiments of the present invention and are not to be construed as limiting the scope thereof.

### Example I

Experiments will be conducted with hypothesis that treatment with rifaximin will improve cognition in subjects having neurodegeneration (e.g., AD). Experiments will be conducted with hypothesis that treatment with rifaximin in subjects having neurodegeneration (e.g., AD) will lower blood ammonia, increase small bowel glutaminase levels, lower circulatory pro-inflammatory cytokines secreted by harmful gut bacteria (e.g., IL-2, IL-4, IL-5, IL-6, IL-8, IL-10, IL-13, tumor necrosis factor alpha), alter serum amyloid-beta 42 levels, lower total-tau levels, alter neurofilament light protein markers, and lower gut microbiota levels.

Such experiments will obtain consent from up to 25 subjects in order to treat 10 eligible subjects with probable mild to moderate AD. The study will be non-randomized. The subjects will be given a therapeutically effective dosage of rifaximin daily for three months (e.g., 550 mg orally twice daily for 3 months) after evaluation to ensure they have no contraindications. The rifaximin dosage and route of administration are the same as that used to treat hepatic encephalopathy, for which the drug and dosage are FDA approved. The dose will remain the same throughout the three-month study of rifaximin administration unless stopped for safety reasons. The age and study sample of mild to moderate probable AD is similar to that used in AD prior trials.

Consent, demographics, history, list of current medications, clinical and safety assessments, MMSE and blood tests (CBC, CMP, TSH, B-12 and Folic acid) will be performed prior to treatment onset. Medical history, physical exam, serum neuronal biomarkers and cytokines, serum ammonia level, adverse events and cognitive testing (ADAS-Cog-11) will be performed prior to treatment onset. A stool sample collection kit will also be given to the subject and caregiver prior to treatment onset, with instructions for use and how to get the sample back to the research coordinator. The stool sample will be obtained prior the subject taking the study drug. Medical history, physical exam, serum neuronal biomarkers and cytokines, serum ammonia level, adverse effects and cognitive testing (ADAS-Cog-11) will be performed at the three-month endpoint. A stool sample collection kit will also be given to the subject and caregiver at the three-month endpoint, with instructions for use and how to get the sample back to the research coordinator.

Such experiments are designed to provide preliminary evidence on the clinical efficacy of rifaximin in improving cognition in AD patients. We will be looking for improvement in test scores, changes in serum levels of neuronal markers and cytokines and changes in gut microbiota following treatment. We will also be collecting data regarding the safety of long-term use of this non-absorbed antibiotic for this disease.

Subjects who meet the following criteria will be considered eligible to participate in the clinical study: Probable Alzheimer's disease (National Institute of Neurological Disorders and Stroke (NINDS) criteria), mild to moderate severity; ages 55-85 both genders; Mini Mental State Exam (MMSE) scores 10-22; Willing and able to comply with all scheduled clinic visits; Stable medical health; Has a family or professional caregiver who has regular contact with subject; Ability to consent or legal guardian who can consent; Living at home or in a facility; On no AD therapies or on stable (2 months) concurrent AD therapies.

Subjects who meet one or more of the following criteria will not be considered eligible to participate in the clinical study: Past history of C diff infection; Assessment, laboratory examination, physical examination or any other medical condition or circumstance making the volunteer unsuitable for participation in the study in the judgment of the study clinicians; Allergy to Rifaximin; Antibiotic use in the last 6 months; Hospitalization in the last 6 months; Are taking medications that interact with Rifaximin; Are taking Cyclosporine; Past or current history of bloody stools or C Diff infection; Elevated LFTs; Clinically significant abnormal hepatic or renal function; Uncorrected thyroid or B12 abnormalities; Participation in another investigational drug trial in the past 30 days; History of febrile illness within 5 days prior to the study period; Hyperammonemia caused by: Valproic acid, Chemotherapy, Lung transplant, Bariatric surgery, Ureterosigmoidoscopy, Hyperalimentation, Urinary tract infection, Errors of metabolism, Urea cycle, Enzyme deficiencies, Organic acidemias, Fatty acid oxidation, Amino acid transport defects.

Experiments will be conducted with hypothesis that treatment with one or more of a blood-ammonia-lowering agent selected from sodium benzoate, sodium phenylacetate, glycerol phenylbutyrate, ornithine phenylacetate, AST-120 (spherical carbon adsorbent), and polyethylene glycol, or any derivatives, salts and esters thereof will improve cognition in subjects having neurodegeneration (e.g., AD). Experiments will be conducted with hypothesis that treatment with such an agent in subjects having neurodegeneration (e.g., AD) will lower blood ammonia, lower tumor necrosis factor alpha), alter serum amyloid-beta 42 levels, lower total-tau levels, and alter neurofilament light protein markers.

Experiments will be conducted with hypothesis that one or more pro-inflammatory bacteria in the colon will be identified as a cause of neurodegeneration and that treatment with a specific antibiotic targeted against that/those bacteria will result in improved cognition in subjects having neurodegeneration (e.g. AD), and will lower circulatory pro-inflammatory cytokines secreted by harmful gut bacteria (e.g., IL-2, IL-4, IL-5, IL-6, IL-8, IL-10, IL-13, tumor necrosis factor alpha), alter serum amyloid-beta 42 levels, lower total-tau levels, alter neurofilament light protein markers, and lower gut microbiota levels.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A method of treating, preventing and/or ameliorating symptoms of a neurodegenerative disorder in a mammal in need thereof, the method comprising administering to the mammal an effective amount of an agent capable of one or more of lowering blood ammonia, increasing small bowel glutaminase levels, lowering circulatory pro-inflammatory cytokines secreted by harmful gut bacteria (e.g., IL-2, IL-4, IL-5, IL-6, IL-8, IL-10, IL-13, tumor necrosis factor alpha), altering serum amyloid-beta 42 levels, lowering total-tau levels, altering neurofilament light protein markers, and lowering gut microbiota levels.

2. The method of claim 1, wherein the neurodegenerative disorder is selected from AD, Parkinson's disease, Huntington's disease, amyotrophic lateral sclerosis, motor neuron disease.

3. The method of claim 1, wherein the mammal is a human patient.

4. The method of claim 1, wherein the agent is a pharmaceutical composition comprising a non-systematically absorbed antibiotic.

5. The method of claim 4, wherein the agent is selected from rifaximin, sodium benzoate, sodium phenylacetate, glycerol phenylbutyrate, ornithine phenylacetate, AST-120 (spherical carbon adsorbent), and polyethylene glycol, or any derivatives, salts and esters thereof

6. A method for one or more of lowering blood and brain ammonia levels in a subject, increasing small bowel glutaminase levels in the subject, lowering circulatory pro-inflammatory cytokines secreted by harmful gut bacteria (e.g., IL-2, IL-4, IL-5, IL-6, IL-8, IL-10, IL-13, tumor necrosis factor alpha) in the subject, altering serum amyloid-beta 42 levels in the subject, lowering total-tau levels in the subject, altering neurofilament light protein markers in the subject, and lowering gut microbiota levels in the subject, comprising administering to the subject a therapeutically effective amount of one or more agents selected from rifaximin, sodium benzoate, sodium phenylacetate, glycerol phenylbutyrate, ornithine phenylacetate, AST-120 (spherical carbon adsorbent), and polyethylene glycol, or any derivatives, salts and esters thereof, wherein the subject is suffering from a neurodegenerative disorder.

7. The method of claim 6, wherein the neurodegenerative disorder is selected from AD, Parkinson's disease, Huntington's disease, amyotrophic lateral sclerosis, motor neuron disease.

8. The method of claim 6, wherein the subject is a human subject.

9-14. (canceled)

15. A method of preventing the onset of a neurodegenerative disorder comprising administering to a subject a therapeutically effective amount of an agent capable of lowering blood and brain ammonia levels in the subject such that the onset of the neurodegenerative disorder is prevented.

16. The method of claim 15, wherein the neurodegenerative disorder is selected from AD, Parkinson's disease, Huntington's disease, amyotrophic lateral sclerosis, motor neuron disease.

17. The method of claim 15, wherein the agent is a pharmaceutical composition comprising a non-systematically absorbed antibiotic.

18. The method of claim 17, wherein the agent is selected from rifaximin, sodium benzoate, sodium phenylacetate, glycerol phenylbutyrate, ornithine phenylacetate, AST-120 (spherical carbon adsorbent), and polyethylene glycol, or any derivatives, salts and esters thereof

19-20. (canceled)
